(19) United States
(12) Patent Application Publication
(10) Pub. No.: US 2024/0087113 A1
(43) Pub. Date: Mar. 14, 2024

(54) Recording Medium, Learning Model Generation Method, and Support Apparatus

(71) Applicant: Anaut Inc., Tokyo (JP)

(72) Inventors: Nao Kobayashi, Tokyo (JP); Yuta Kumazu, Tokyo (JP); Seigo Senya, Tokyo (JP)

(21) Appl. No.: 18/272,328

(22) PCT Filed: Jan. 18, 2022
(86) PCT No.: PCT/JP2022/001623

§ 371 Date: Jul. 13, 2023

(30) Foreign Application Priority Data
Jan. 19, 2021 (JP) 2021-006535

(51) Int. Cl. G06T 7/00; A61B 1/00

(52) U.S. Cl. CPC G06T 7/0012; A61B 1/000094; G06T 2207/30024; G06T 2207/30101

(57) ABSTRACT

A computer program causing a computer to execute processing includes acquiring an operative field image obtained by imaging an operative field of scopic surgery, and recognizing a target tissue portion included in the acquired operative field image so as to be distinguished from a blood vessel tissue portion appearing on a surface of the target tissue portion by using a learning model trained to output information regarding a target tissue when the operative field image is input.

### CROSS-REFERENCE TO RELATED APPLICATIONS

This application is the national phase under 35 U. S. C. § 371 of PCT International Application No. PCT/JP2022/001623 which has an International filing date of Jan. 18, 2022 and designated the United States of America.

### FIELD

The present invention relates to a recording medium, a learning model generation method, and a support apparatus.

### BACKGROUND

In laparoscopic surgery, for example, surgery to remove a lesion such as a malignant tumor formed in the patient's body is performed.

At this time, the inside of the patient's body is imaged by a laparoscope, and the obtained operative field image is displayed on a monitor (see Japanese Patent Application Laid-Open No. 2005-287839, for example).

Conventionally, it has been difficult to recognize tissues such as nerves and ureters that require the operator's attention from the operative field image and provide notification to the operator.

### SUMMARY

It is an object of the present application to provide a recording medium, a learning model generation method, and a support apparatus capable of outputting the recognition results of tissues such as nerves and ureters from an operative field image.

A recording medium according to one aspect of the present application is a non-transitory computer readable recording medium storing a computer program that causes a computer to execute processing includes acquiring an operative field image obtained by imaging an operative field of scopic surgery; and recognizing a target tissue portion included in the acquired operative field image so as to be distinguished from a blood vessel tissue portion appearing on a surface of the target tissue portion by using a learning model trained to output information regarding a target tissue when the operative field image is input.

A learning model generation method according to one aspect of the present application includes: causing a computer to acquire training data including an operative field image obtained by imaging an operative field of scopic surgery and correct data in which a target tissue portion included in the operative field image is labeled so as to be distinguished from a blood vessel tissue portion appearing on a surface of the target tissue portion; and causing the computer to generate a learning model that outputs information regarding a target tissue based on the acquired set of training data when the operative field image is input.

A support apparatus according to one aspect of the present application includes: an acquisition unit that acquires an operative field image obtained by imaging an operative field of scopic surgery; a recognition unit that recognizes a target tissue portion included in the acquired operative field image so as to be distinguished from a blood vessel tissue portion appearing on a surface of the target tissue portion by using a learning model trained to output information regarding a target tissue when the operative field image is input; and an output unit that outputs support information regarding the scopic surgery based on a recognition result of the recognition unit.

According to the present application, it is possible to output the recognition results of tissues, such as nerves and ureters, from the operative field image.

The above and further objects and features of the invention will more fully be apparent from the following detailed description with accompanying drawings.

### BRIEF DESCRIPTION OF THE DRAWINGS

FIG. 1 is a schematic diagram illustrating the schematic configuration of a laparoscopic surgery support system according to a first embodiment.

FIG. 2 is a block diagram illustrating the internal configuration of a support apparatus.

FIG. 3 is a schematic diagram depicting an example of an operative field image.

FIG. 4 is a schematic diagram depicting a configuration example of a learning model.

FIG. 5 is a schematic diagram depicting a recognition result of the learning model.

FIG. 6 is a partial enlarged view depicting a recognition result in the first embodiment.

FIG. 7 is a partial enlarged view depicting a recognition result in a comparative example.

FIG. 8 is a flowchart illustrating a generation procedure of a learning model.

FIG. 9 is a flowchart illustrating the execution procedure of surgery support.

FIG. 10 is a schematic diagram depicting a display example of a display device.

FIG. 11 is a schematic diagram depicting an example of an operative field image in a second embodiment.

FIG. 12 is an explanatory diagram illustrating the configuration of a learning model according to the second embodiment.

FIG. 13 is a schematic diagram depicting a display example of a recognition result in the second embodiment.

FIG. 14 is a schematic diagram depicting an example of an operative field image in a third embodiment.

FIG. 15 is an explanatory diagram illustrating the configuration of a learning model according to the third embodiment.

FIG. 16 is a schematic diagram depicting a display example of a recognition result in the third embodiment.

FIG. 17 is a schematic diagram depicting a display example in a fourth embodiment.

FIG. 18 is an explanatory diagram illustrating a display method according to a fifth embodiment.

FIG. 19 is a flowchart illustrating the procedure of processing performed by a support apparatus according to a sixth embodiment.

FIG. 20 is a schematic diagram depicting a display example in the sixth embodiment.

FIG. 21 is an explanatory diagram illustrating the configuration of a learning model according to a seventh embodiment.

FIG. 22 is a schematic diagram depicting a display example of a recognition result in the seventh embodiment.

FIG. 23 is an explanatory diagram illustrating the configuration of a learning model according to an eighth embodiment.

FIG. 24 is an explanatory diagram illustrating a method of specifying an organ boundary.

FIG. 25 is a flowchart illustrating the procedure of processing performed by a support apparatus according to the eighth embodiment.

### DESCRIPTION OF EMBODIMENTS

Hereinafter, a form in which the present invention is applied to a support system for laparoscopic surgery will be specifically described with reference to the diagrams. In addition, the present invention is not limited to laparoscopic surgery, and can be applied to scopic surgery in general using an imaging apparatus, such as a thoracoscope, a gastrointestinal endoscope, a cystoscope, an arthroscope, a robot-assisted endoscope, a spine endoscope, a surgical microscope, a neuroendoscope, and an outer scope.

### First Embodiment

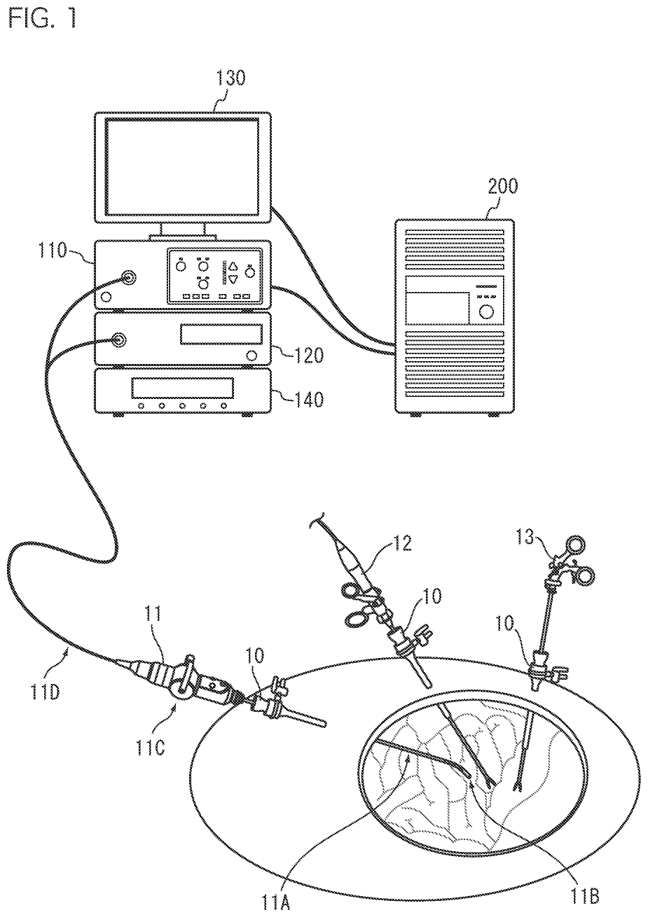

FIG. 1 is a schematic diagram illustrating the schematic configuration of a laparoscopic surgery support system according to a first embodiment. In laparoscopic surgery, instead of performing an abdominal operation, a plurality of piercing instruments called trocars 10 are attached to the patient's abdominal wall, and instruments such as a laparoscope 11, an energy treatment instrument 12, and forceps 13 are inserted into the patient's body through the openings provided in the trocars 10. The operator performs a treatment, such as excision of the affected area, using the energy treatment instrument 12 while viewing an image of the inside of the patient's body (operative field image) captured by the laparoscope 11 in real time. Surgical instruments such as the laparoscope 11, the energy treatment instrument 12, and the forceps 13 are held by an operator, a robot, or the like. The operator is a medical worker involved in laparoscopic surgery, and includes a surgeon, an assistant, a nurse, a doctor who monitors the surgery, and the like.

The laparoscope 11 includes an insertion portion 11A to be inserted into the patient's body, an imaging apparatus 11B built in the distal end portion of the insertion portion 11A, an operation portion 11C provided in the rear end portion of the insertion portion 11A, and a universal cord 11D for connection to a camera control unit (CCU) 110 or a light source device 120.

The insertion portion 11A of the laparoscope 11 is formed of a rigid tube. A bending portion is provided at the distal end portion of the rigid tube. A bending mechanism in the bending portion is a known mechanism built in a general laparoscope, and is configured to bend in four directions, for example, up, down, left, and right by pulling an operation wire linked to the operation of the operation portion 11C. In addition, the laparoscope 11 is not limited to a flexible scope having the bending portion described above, and may be a rigid scope that does not have a bending portion.

The imaging apparatus 11B includes a driver circuit including a solid-state imaging device such as a CMOS (Complementary Metal Oxide Semiconductor), a timing generator (TG), an analog front end (AFE), and the like. The driver circuit of the imaging apparatus 11B acquires RGB color signals output from the solid-state imaging device in synchronization with a clock signal output from the TG, and performs necessary processing, such as noise removal, amplification, and AD conversion, in the AFE to generate image data in a digital form. The driver circuit of the imaging apparatus 11B transmits the generated image data to the CCU 110 through the universal cord 11D.

The operation portion 11C includes an angle lever, a remote switch, and the like that are operated by the operator. The angle lever is an operation tool that receives an operation for bending the bending portion. A bending operation knob, a joystick, or the like may be provided instead of the angle lever. Examples of the remote switch include a selector switch for switching between moving image display and still image display of an observation image and a zoom switch for enlarging or reducing the observation image. A specific function set in advance may be assigned to the remote switch, or a function set by the operator may be assigned to the remote switch.

In addition, a vibrator configured by a linear resonance actuator, a piezo actuator, or the like may be built in the operation portion 11C. When an event of which the operator who operates the laparoscope 11 is to be notified occurs, the CCU 110 may vibrate the operation portion 11C by activating the vibrator built in the operation portion 11C to notify the operator of the occurrence of the event.

A transmission cable for transmitting a control signal output from the CCU 110 to the imaging apparatus 11B or image data output from the imaging apparatus 11B, a light guide for guiding illumination light emitted from the light source device 120 to the distal end portion of the insertion portion 11A, and the like are arranged inside the insertion portion 11A, the operation portion 11C, and the universal cord 11D of the laparoscope 11. The illumination light emitted from the light source device 120 is guided to the distal end portion of the insertion portion 11A through the light guide, and is emitted to the operative field through an illumination lens provided at the distal end portion of the insertion portion 11A. In addition, although the light source device 120 is described as an independent device in the present embodiment, the light source device 120 may be built in the CCU 110.

The CCU 110 includes a control circuit for controlling the operation of the imaging apparatus 11B provided in the laparoscope 11, an image processing circuit for processing the image data from the imaging apparatus 11B input through the universal cord 11D, and the like. The control circuit includes a CPU (Central Processing Unit), a ROM (Read Only Memory), a RAM (Random Access Memory), and the like, and controls imaging start, imaging stop, zooming, and the like by outputting a control signal to the imaging apparatus 11B in response to the operations of various switches provided in the CCU 110 or the operation of the operation portion 11C provided in the laparoscope 11. The image processing circuit includes a DSP (Digital Signal Processor), an image memory, and the like, and performs appropriate processing, such as color separation, color interpolation, gain correction, white balance adjustment, and gamma correction, on the image data input through the universal cord 11D. The CCU 110 generates frame images for a moving image from the image data after processing, and sequentially outputs the generated frame images to a support apparatus 200, which will be described later. The frame rate of frame images is, for example, 30 FPS (Frames Per Second).

The CCU 110 may generate video data conforming to a predetermined standard, such as NTSC (National Television System Committee), PAL (Phase Alternating Line), and DICOM (Digital Imaging and COmmunication in Medicine). By outputting the generated video data to a display device 130, the CCU 110 can display an operative field image (video) on the display screen of the display device 130 in real time. The display device 130 is a monitor including a liquid crystal panel, an organic EL (Electro-Luminescence) panel, or the like. In addition, the CCU 110 may output the generated video data to an image recording device 140 so that the image recording device 140 records the video data. The image recording device 140 includes a recording device such as an HDD (Hard Disk Drive) that records video data output from the CCU 110 together with an identifier for identifying each surgery, surgery date and time, surgery location, patient name, operator name, and the like.

The support apparatus 200 generates support information related to the laparoscopic surgery based on the image data input from the CCU 110 (that is, image data of an operative field image obtained by imaging the operative field). Specifically, the support apparatus 200 performs processing for recognizing a target tissue to be recognized and a blood vessel tissue (surface blood vessel) appearing on the surface of the target tissue so as to be distinguished from each other and displaying information regarding the recognized target tissue on the display device 130. In the first to sixth embodiments, a configuration in which a nerve tissue is recognized as a target tissue will be described. In a seventh embodiment, which will be described later, a configuration in which a ureter tissue is recognized as a target tissue will be described. The target tissue is not limited to the nerve tissue or the ureter tissue, and may be any organ including surface blood vessels, such as arteries, vas deferens, bile ducts, bones, and muscles.

In the present embodiment, a configuration will be described in which nerve tissue recognition processing is performed by the support apparatus 200. However, the CCU 110 may be made to have the same function as the support apparatus 200, and the CCU 110 may perform the nerve tissue recognition processing.

Hereinafter, the internal configuration of the support apparatus 200 and recognition processing and display processing performed by the support apparatus 200 will be described.

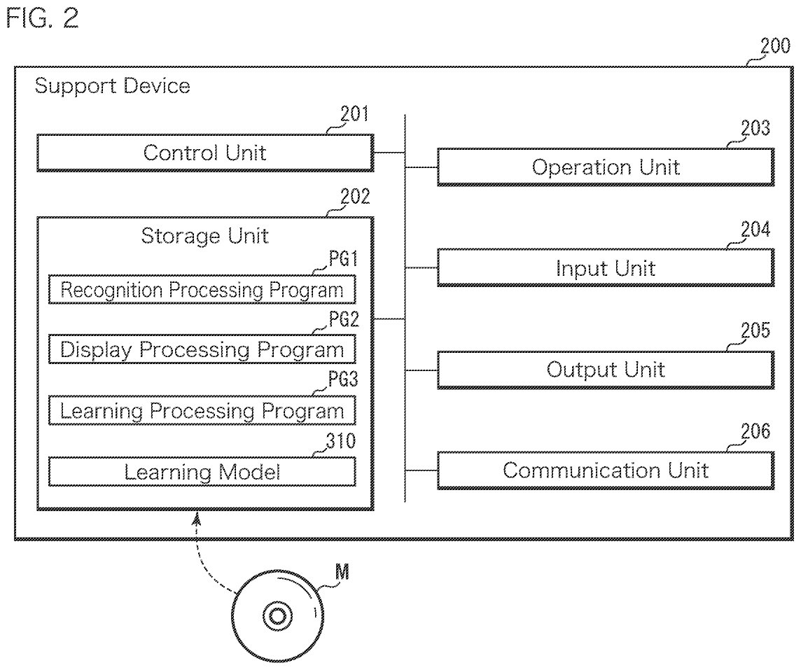

FIG. 2 is a block diagram illustrating the internal configuration of the support apparatus 200. The support apparatus 200 is a dedicated or general-purpose computer including a control unit 201, a storage unit 202, an operation unit 203, an input unit 204, an output unit 205, a communication unit 206, and the like. The support apparatus 200 may be a computer provided inside the operating room, or may be a computer provided outside the operating room. In addition, the support apparatus 200 may be a server provided in a hospital where laparoscopic surgery is performed, or may be a server provided outside the hospital.

The control unit 201 includes, for example, a CPU, a ROM, and a RAM. The ROM provided in the control unit 201 stores a control program and the like for controlling the operation of each hardware unit provided in the support apparatus 200. The CPU in the control unit 201 controls the operation of each hardware unit by executing the control program stored in the ROM or various computer programs stored in the storage unit 202, which will be described later, so that the entire apparatus functions as a support apparatus in the present application. The RAM provided in the control unit 201 temporarily stores data and the like that are used during the execution of arithmetic operations.

In the present embodiment, the control unit 201 is configured to include a CPU, a ROM, and a RAM. However, the control unit 201 may have any configuration. For example, an arithmetic circuit or a control circuit including one or more GPUs (Graphics Processing Unit), one or more quantum processors, one or more volatile memories or non-volatile memories, and the like may be used. In addition, the control unit 201 may have functions such as a clock that outputs date and time information, a timer that measures the elapsed time from when a measurement start instruction is given until a measurement end instruction is given, and a counter for number counting.

The storage unit 202 includes a storage device using a hard disk, a flash memory, or the like. The storage unit 202 stores computer programs executed by the control unit 201, various kinds of data acquired from the outside, various kinds of data generated inside the apparatus, and the like.

The computer programs stored in the storage unit 202 include a recognition processing program PG1 that causes the control unit 201 to perform processing for recognizing a target tissue portion included in the operative field image so as to be distinguished from a blood vessel tissue portion, a display processing program PG2 that causes the control unit 201 to perform processing for displaying support information based on the recognition result on the display device 130, and a learning processing program PG3 for generating a learning model 310. In addition, the recognition processing program PG1 and the display processing program PG2 do not need to be independent computer programs, and may be implemented as one computer program. These programs are provided, for example, by a non-temporary recording medium M in which the computer programs are recorded in a readable manner. The recording medium M is a portable memory such as a CD-ROM, a USB memory, and an SD (Secure Digital) card. The control unit 201 reads a desired computer program from the recording medium M by using a reader (not depicted), and stores the read computer program in the storage unit 202. Alternatively, the computer program may be provided by communication using the communication unit 206.

In addition, the storage unit 202 stores the learning model 310 used in the recognition processing program PG1 described above. The learning model 310 is a learning model trained so as to output a recognition result related to the target tissue in response to the input of the operative field image. The learning model 310 is described by its definition information. The definition information of the learning model 310 includes parameters such as information of layers included in the learning model 310, information of nodes forming each layer, and weighting and biasing between nodes. These parameters are trained by using a predetermined learning algorithm with an operative field image obtained by imaging the operative field and correct data, which indicates the target tissue portion in the operative field image, as training data. The configuration and generation procedure of the learning model 310 will be detailed later.

The operation unit 203 includes operation devices such as a keyboard, a mouse, a touch panel, a non-contact panel, a stylus pen, and a voice input using a microphone. The operation unit 203 receives an operation by an operator or the like, and outputs information regarding the received operation to the control unit 201. The control unit 201 performs appropriate processing according to the operation information input from the operation unit 203. In addition, in the present embodiment, the support apparatus 200 is configured to include the operation unit 203, but may be configured to receive operations through various devices such as the CCU 110 connected to the outside.

The input unit 204 includes a connection interface for connection to an input device. In the present embodiment, the input device connected to the input unit 204 is the CCU 110. The input unit 204 receives image data of an operative field image captured by the laparoscope 11 and processed by the CCU 110. The input unit 204 outputs the input image data to the control unit 201. In addition, the control unit 201 may store the image data acquired from the input unit 204 in the storage unit 202.

The output unit 205 includes a connection interface for connection to an output device. In the present embodiment, the output device connected to the output unit 205 is the display device 130. When generating information of which the operator or the like is to be notified, such as the recognition result of the learning model 310, the control unit 201 outputs the generated information to the display device 130 through the output unit 205 to display the information on the display device 130. In the present embodiment, the display device 130 is connected to the output unit 205 as an output device. However, an output device such as a speaker that outputs sound may be connected to the output unit 205.

The communication unit 206 includes a communication interface for transmitting and receiving various kinds of data. The communication interface provided in the communication unit 206 is a communication interface conforming to a wired or wireless communication standard used in Ethernet (registered trademark) or WiFi (registered trademark). When data to be transmitted is input from the control unit 201, the communication unit 206 transmits the data to be transmitted to a designated destination. In addition, when data transmitted from an external device is received, the communication unit 206 outputs the received data to the control unit 201.

The support apparatus 200 does not need to be a single computer, and may be a computer system including a plurality of computers or peripheral devices. In addition, the support apparatus 200 may be a virtual machine that is virtually constructed by software.

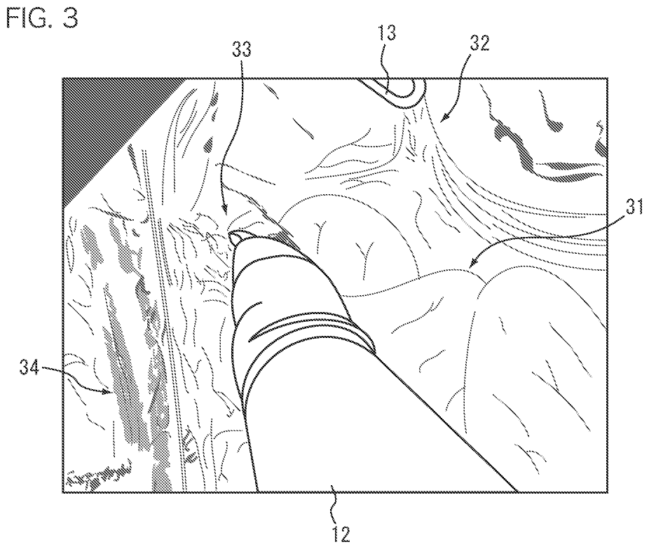

Next, the operative field image input to the support apparatus 200 will be described. FIG. 3 is a schematic diagram depicting an example of the operative field image. The operative field image in the present embodiment is an image obtained by imaging the inside of the patient's abdominal cavity with the laparoscope 11. The operative field image does not need to be a raw image output from the imaging apparatus 11B of the laparoscope 11, and may be an image (frame image) processed by the CCU 110 or the like.

The operative field imaged by the laparoscope 11 includes tissues forming organs, blood vessels, nerves, and the like, connective tissues present between tissues, tissues including lesions such as tumors, and tissues such as membranes or layers covering tissues. The operator dissects a tissue including a lesion by using an instrument, such as forceps and an energy treatment instrument, while checking the relationship between these anatomical structures. The operative field image depicted as an example in FIG. 3 depicts a scene in which a membrane 32 covering an organ 31 is pulled by using the forceps 13 and a tissue including a lesion 33 is dissected by using the energy treatment instrument 12. In addition, in the example of FIG. 3, it is depicted how a nerve 34 runs in the vertical direction in the diagram near the lesion 33. In scopic surgery, when the nerve is damaged during traction or dissection, postoperative dysfunction may occur. For example, damage to the hypogastric nerve in colon surgery can cause dysuria. In addition, damage to the semicircular nerve during esophagectomy or pulmonary resection can cause dysphagia.

In order to avoid nerve damage during surgery, it is important to check the running direction of nerves. However, nerves are rarely completely exposed and often overlap other tissues, such as blood vessels. For this reason, it is not always easy for the operator to check the running direction of the nerves. Therefore, the support apparatus 200 according to the present embodiment recognizes a nerve tissue portion included in the operative field image so as to be distinguished from a blood vessel tissue portion, and outputs support information related to the laparoscopic surgery based on the recognition result, by using the learning model 310.

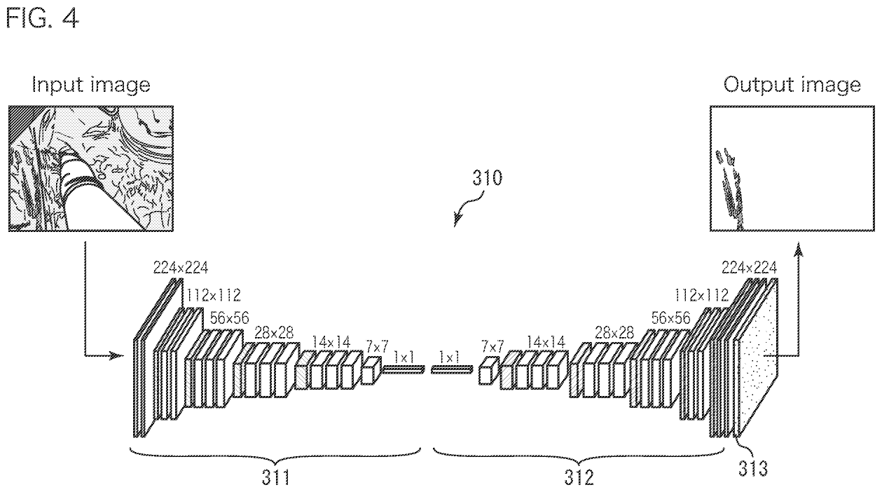

Hereinafter, a configuration example of the learning model 310 will be described. FIG. 4 is a schematic diagram depicting a configuration example of the learning model 310. The learning model 310 is a learning model for performing image segmentation, and is constructed by a neural network having a convolutional layer such as SegNet, for example. The learning model 310 is not limited to SegNet, and may be constructed by using any neural network that can perform image segmentation, such as FCN (Fully Convolutional Network), U-Net (U-Shaped Network), PSPNet (Pyramid Scene Parsing Network). In addition, the learning model 310 may be constructed by using a neural network for object detection, such as YOLO (You Only Look Once) or SSD (Single Shot Multi-Box Detector), instead of the neural network for image segmentation.

In the present embodiment, the input image for the learning model 310 is an operative field image obtained from the laparoscope 11. The learning model 310 is trained so as to output an image depicting the recognition result of the nerve tissue included in the operative field image in response to the input of the operative field image.

The learning model 310 includes, for example, an encoder 311, a decoder 312, and a softmax layer 313. The encoder 311 is configured by alternately arranging convolution layers and pooling layers. The convolution layers are multi-layered into two to three layers. In the example of FIG. 4, the convolutional layers are depicted without hatching, and the pooling layers are depicted with hatching.

In the convolution layer, a convolution operation between the input data and a filter having each predetermined size (for example, 3×3 or 5×5) is performed. That is, an input value input to the position corresponding to each element of the filter is multiplied by a weighting factor set in advance in the filter for each element, and the linear sum of the multiplication values for these elements is calculated. The output in the convolutional layer is obtained by adding the set bias to the calculated linear sum. In addition, the result of the convolution operation may be transformed by an activation function. For example, ReLU (Rectified Linear Unit) can be used as the activation function. The output of the convolutional layer represents a feature map in which the features of the input data are extracted.

In the pooling layer, the local statistic of the feature map output from the convolutional layer, which is an upper layer connected to the input side, is calculated. Specifically, a window having a predetermined size (for example, 2×2 or 3×3) corresponding to the position of the upper layer is set, and the local statistic is calculated from the input values within the window. For example, a maximum value can be used as the statistic. The size of the feature map output from the pooling layer is reduced (downsampled) according to the size of the window. In the example of FIG. 4, it is depicted that the encoder 311 sequentially repeats the operation in the convolution layer and the operation in the pooling layer to sequentially downsample the input image of 224 pixels×224 pixels to feature maps of 112×112, 56×56, 28×28, . . . , 1×1.

The output (feature map of 1×1 in the example of FIG. 4) of the encoder 311 is input to the decoder 312. The decoder 312 is configured by alternately arranging deconvolution layers and depooling layers. The deconvolution layers are multi-layered into two to three layers. In the example of FIG. 4, the deconvolution layers are depicted without hatching, and the depooling layers are depicted with hatching.

In the deconvolution layer, a deconvolution operation is performed on the input feature map. The deconvolution operation is an operation to restore the feature map before the convolution operation under the presumption that the input feature map is a result of the convolution operation using a specific filter. In this operation, when a specific filter is represented by a matrix, a product of a transposed matrix for this matrix and the input feature map is calculated to generate a feature map for output. In addition, the operation result of the deconvolution layer may be transformed by an activation function such as ReLU described above.

The depooling layers of the decoder 312 are individually mapped in a one-to-one manner to the pooling layers of the encoder 311, and a pair thereof correspond to each other have substantially the same size. The depooling layer again enlarges (upsamples) the size of the feature map downsampled in the pooling layer of the encoder 311. In the example of FIG. 4, it is depicted that the decoder 312 sequentially repeats the operation in the convolution layer and the operation in the pooling layer for sequential upsampling to feature maps of 1×1, 7×7, 14×14, . . . , 224×224.

The output (feature map of 224×224 in the example of FIG. 4) of the decoder 312 is input to the softmax layer 313. The softmax layer 313 outputs the probability of a label for identifying a part at each position (pixel) by applying a softmax function to the input value from the deconvolution layer connected to the input side. In the present embodiment, a label for identifying the nerve tissue may be set to identify whether or not a part belongs to the nerve tissue in units of pixels. By extracting pixels for which the probability of the label output from the softmax layer 313 is equal to or greater than a threshold value (for example, 70% or more), an image depicting the recognition result (hereinafter, referred to as a recognition image) of the nerve tissue portion is obtained.

In addition, in the example of FIG. 4, the image of 224 pixels×224 pixels is used as an input image for the learning model 310. However, the size of the input image is not limited to the above, and can be appropriately set according to the processing capacity of the support apparatus 200, the size of the operative field image obtained from the laparoscope 11, and the like. In addition, the input image for the learning model 310 does not need to be the entire operative field image obtained from the laparoscope 11, and may be a partial image generated by cutting out a region of interest of the operative field image. Since the region of interest including a treatment target is often located near the center of the operative field image, for example, a partial image obtained by cutting out a rectangle near the center of the operative field image so that the size is about half of the original size may be used. By reducing the size of the image input to the learning model 310, it is possible to improve the recognition accuracy while increasing the processing speed.

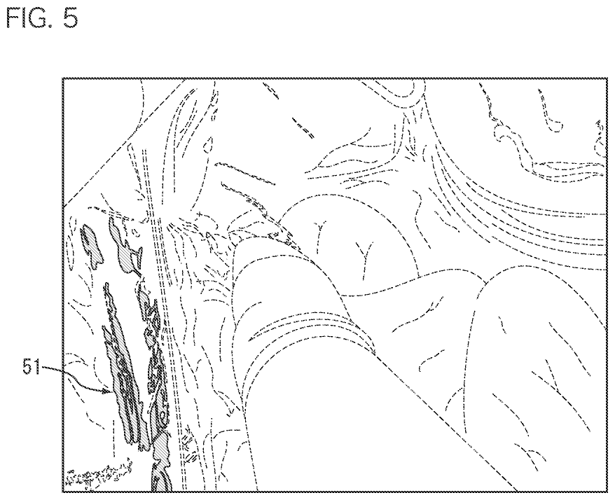

FIG. 5 is a schematic diagram depicting a recognition result of the learning model 310. In the example of FIG. 5, a nerve tissue portion 51 recognized by using the learning model 310 is hatched, and other organs, membranes, and surgical instruments are indicated by dashed lines for reference. The control unit 201 of the support apparatus 200 generates a recognition image of the nerve tissue in order to display the recognized nerve tissue portion in a distinguishable manner. The recognition image is an image which has the same size as the operative field image and in which a specific color is assigned to pixels recognized as the nerve tissue. The color assigned to the nerve tissue is set arbitrarily. For example, the color assigned to the nerve tissue may be a white color similar to nerves, or may be a blue color that is not present inside the human body. In addition, information indicating the degree of transparency is added to each pixel forming the recognition image, and a non-transparent value is set for pixels recognized as the nerve tissue and a transparent value is set for the other pixels. The support apparatus 200 can display the nerve tissue portion as a structure having a specific color on the operative field image by displaying the recognition image generated in this manner so as to be superimposed on the operative field image.

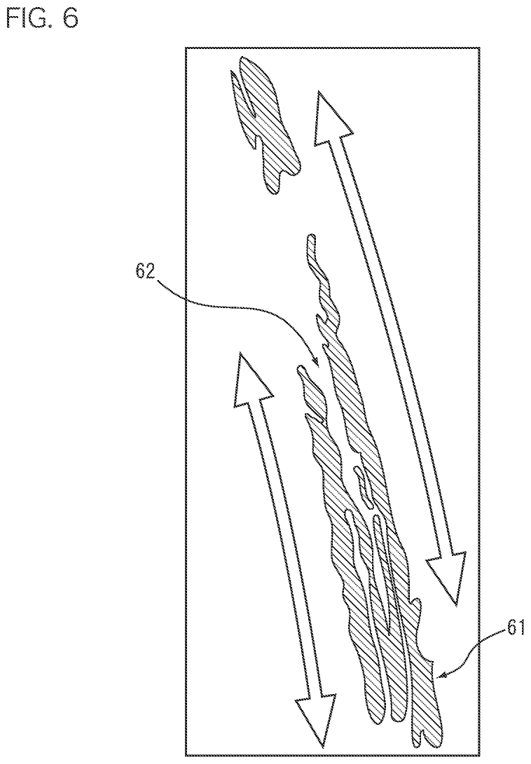

FIG. 6 is a partial enlarged view depicting a recognition result in the first embodiment. FIG. 6 depicts a result of recognizing a nerve tissue portion 61 included in the operative field image and a blood vessel tissue portion 62 appearing on the surface of the nerve tissue portion 61 so as to be distinguished from each other. In the example of FIG. 6, only the recognized nerve tissue portion 61 is hatched, excluding the blood vessel tissue portion 62. From this recognition result, it is possible to check the presence of two nerves running in parallel in the directions of two arrows depicted in the diagram.

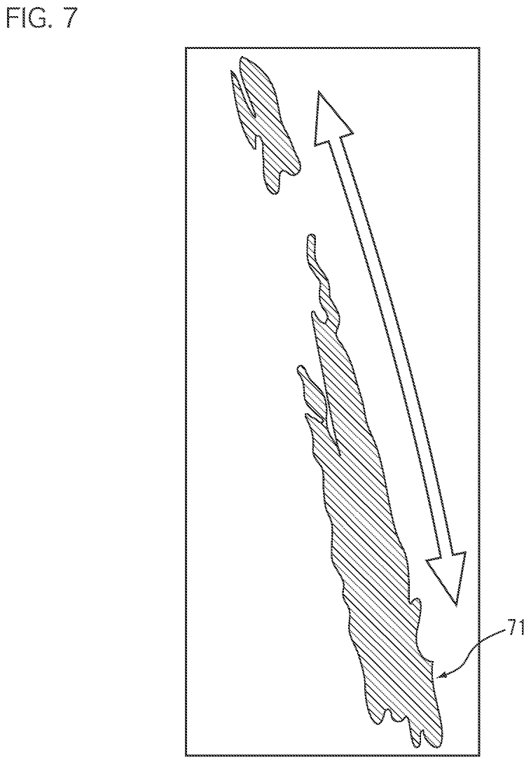

FIG. 7 is a partial enlarged view depicting a recognition result in a comparative example. The comparative example in FIG. 7 depicts a result of recognizing a nerve tissue portion 71 in the same region as in FIG. 6 without distinguishing the nerve tissue portion 71 from the blood vessel tissue portion appearing on the surface. From this recognition result, it is not possible to clearly grasp the presence of two nerves running in parallel. For this reason, it may be interpreted that a relatively thick nerve runs in the direction of the arrow.

In the present embodiment, in order to recognize a nerve tissue portion included in the operative field image and a blood vessel tissue portion appearing on the surface of the nerve tissue portion so as to be distinguished from each other, the learning model 310 that recognizes whether or not each pixel corresponds to the nerve tissue is generated. As a preparatory stage for generating the learning model 310, annotation is performed on the captured operative field image.

In the preparatory stage for generating the learning model 310, the operator (expert such as a doctor) causes the display device 130 to display an operative field image recorded in the image recording device 140, and performs annotation by designating a portion corresponding to the nerve tissue in units of pixels using a mouse, a stylus pen, or the like provided as the operation unit 203. At this time, it is preferable that the operator designates a portion corresponding to the nerve tissue in units of pixels, excluding the blood vessel tissue appearing on the surface of the nerve tissue. A set of a large number of operative field images used for annotation and data (correct data) indicating the positions of pixels corresponding to the nerve tissue designated in each operative field image is stored in the storage unit 202 of the support apparatus 200 as training data for generating the learning model 310. In order to increase the number of pieces of training data, the training data may include a set of operative field images generated by applying perspective transformation, reflection processing, and the like and correct data for the operative field image. In addition, as the training progresses, the training data may include a set of the operative field image and the recognition result (correct data) of the learning model 310 obtained by inputting the operative field image.

In addition, when performing annotation, the operator may label pixels corresponding to the blood vessel tissue to be excluded as incorrect data. A set of operative field images used for annotation, data (correct data) indicating the positions of pixels corresponding to the nerve tissue designated in each operative field image, and data (incorrect data) indicating the positions of pixels corresponding to the designated blood vessel tissue may be stored in the storage unit 202 of the support apparatus 200 as training data for generating the learning model 310.

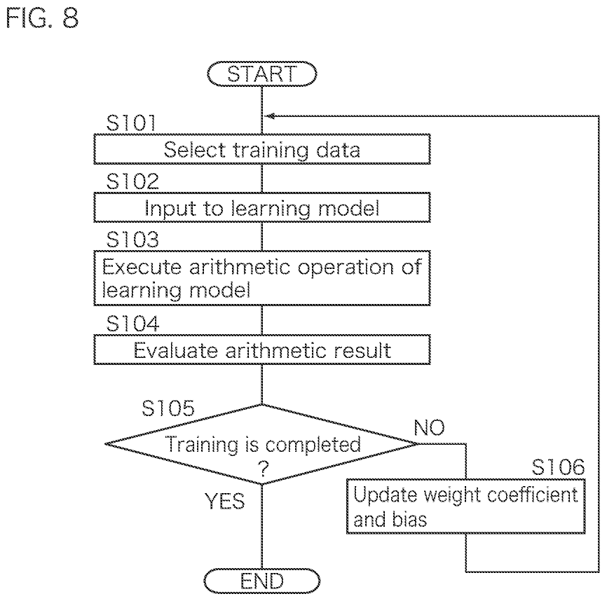

The support apparatus 200 generates the learning model 310 by using the training data described above. FIG. 8 is a flowchart illustrating a generation procedure of the learning model 310. The control unit 201 of the support apparatus 200 reads the learning processing program PG3 from the storage unit 202 and executes the following procedure to generate the learning model 310. In addition, it is assumed that the definition information describing the learning model 310 has initial values in a stage before the training is started.

The control unit 201 accesses the storage unit 202 and selects a set of training data from training data prepared in advance to generate the learning model 310 (step S101). The control unit 201 inputs an operative field image included in the selected training data to the learning model 310 (step S102), and executes the arithmetic operation of the learning model 310 (step S103). That is, the control unit 201 generates a feature map from the input operative field image, and executes the arithmetic operation using the encoder 311 that sequentially downsamples the generated feature map, the arithmetic operation using the decoder 312 that sequentially upsamples the feature map input from the encoder 311, and the arithmetic operation using the softmax layer 313 for identifying each pixel of the feature map finally obtained from the decoder 312.

The control unit 201 acquires the arithmetic result from the learning model 310 and evaluates the acquired arithmetic result (step S104). For example, the control unit 201 may evaluate the arithmetic result by calculating the degree of similarity between the nerve tissue image data obtained as the arithmetic result and the correct data included in the training data. The degree of similarity is calculated by using, for example, the Jaccard coefficient. The Jaccard coefficient is given by A∩B/A∪B×100(%), where A is a nerve tissue portion extracted by the learning model 310 and B is a nerve tissue portion included in the correct data. Instead of the Jaccard coefficient, a Dice coefficient or a Simpson coefficient may be calculated, or other known methods may be used to calculate the degree of similarity. When the training data includes incorrect data, the control unit 201 may proceed with training by referring to the incorrect data. For example, when the nerve tissue portion extracted by the learning model 310 corresponds to the blood vessel tissue portion included in the incorrect data, the control unit 201 may perform a process of subtracting the degree of similarity.

The control unit 201 determines whether or not training has been completed based on the arithmetic result evaluation (step S105). The control unit 201 can determine that training has been completed when the degree of similarity equal to or greater than a threshold value set in advance is obtained.

When it is determined that training has not been completed (S105: NO), the control unit 201 sequentially updates a weighting factor and a bias in each layer of the learning model 310 from the output side to the input side of the learning model 310 by using a back propagation method (step S106). After updating the weighting factor and the bias of each layer, the control unit 201 returns to step S101 to perform the processes from step S101 to step S105 again.

When it is determined in step S105 that training has been completed (S105: YES), the learning model 310 that has completed training is obtained. Therefore, the control unit 201 ends the process according to this flowchart.

In the present embodiment, the learning model 310 is generated by the support apparatus 200. However, the learning model 310 may be generated by using an external computer such as a server apparatus. In this case, the support apparatus 200 may acquire the learning model 310 generated by the external computer by using means such as communication, and store the acquired learning model 310 in the storage unit 202.

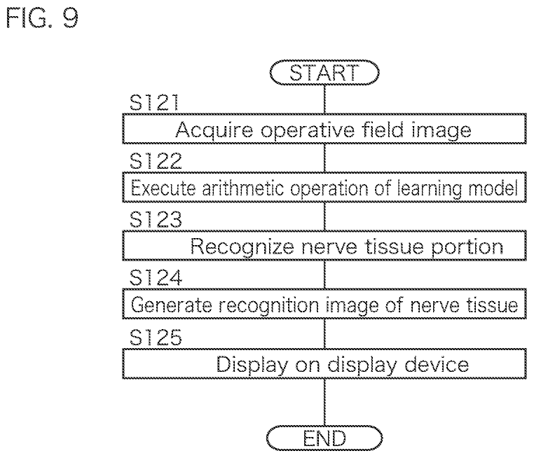

The support apparatus 200 supports surgery in the operation phase after the learning model 310 is generated. FIG. 9 is a flowchart illustrating the execution procedure of surgery support. The control unit 201 of the support apparatus 200 reads the recognition processing program PG1 and the display processing program PG2 from the storage unit 202 and executes these, thereby executing the following procedure. When laparoscopic surgery is started, an operative field image obtained by imaging the operative field with the imaging apparatus 11B of the laparoscope 11 is output to the CCU 110 through the universal cord 11D at any time. The control unit 201 of the support apparatus 200 acquires the operative field image output from the CCU 110 through the input unit 204 (step S121). The control unit 201 performs the following processing each time an operative field image is acquired.

The control unit 201 inputs the acquired operative field image to the learning model 310, executes the arithmetic operation using the learning model 310 (step S122), and recognizes a nerve tissue portion included in the operative field image (step S123). That is, the control unit 201 generates a feature map from the input operative field image, and executes the arithmetic operation using the encoder 311 that sequentially downsamples the generated feature map, the arithmetic operation using the decoder 312 that sequentially upsamples the feature map input from the encoder 311, and the arithmetic operation using the softmax layer 313 for identifying each pixel of the feature map finally obtained from the decoder 312. In addition, the control unit 201 recognizes, as a nerve tissue portion, pixels for which the probability of the label output from the softmax layer 313 is equal to or greater than a threshold value (for example, 70% or more).

When generating the learning model 310, when annotation has been performed so as to recognize a nerve tissue in the operator's central visual field, only the nerve tissue present in the central visual field of the operator is recognized in step S123. In addition, when annotation has been performed so as to recognize a nerve tissue that is not in the operator's central visual field, only the nerve tissue that is not in the operator's central visual field is recognized in step S123. In addition, when annotation has been performed so as to recognize a nerve tissue under tension, recognition as a nerve tissue is performed in a stage when the nerve tissue transitions from the state before being tense to the tense state in step S123. In addition, when annotation has been performed so as to recognize a nerve tissue that has begun to be exposed to the operative field, recognition as a nerve tissue is performed in a stage when the nerve tissue begins to be exposed by pulling or excising the membrane or layer that covers the tissue such as an organ.

The control unit 201 generates a recognition image of the nerve tissue in order to display the nerve tissue portion recognized by using the learning model 310 in a distinguishable manner (step S124). The control unit 201 may assign a specific color, such as a white color similar to nerves or a blue color that is not present inside the human body, to pixels recognized as the nerve tissue and set the degree of transparency so that the background is transparent for pixels other than the nerve tissue.

The control unit 201 outputs the recognition image of the nerve tissue generated in step S124 to the display device 130 through the output unit 205 together with the operative field image acquired in step S121, so that the recognition image is displayed on the display device 130 so as to be superimposed on the operative field image (step S125). As a result, the nerve tissue portion recognized by using the learning model 310 is displayed on the operative field image as a structure having a specific color.

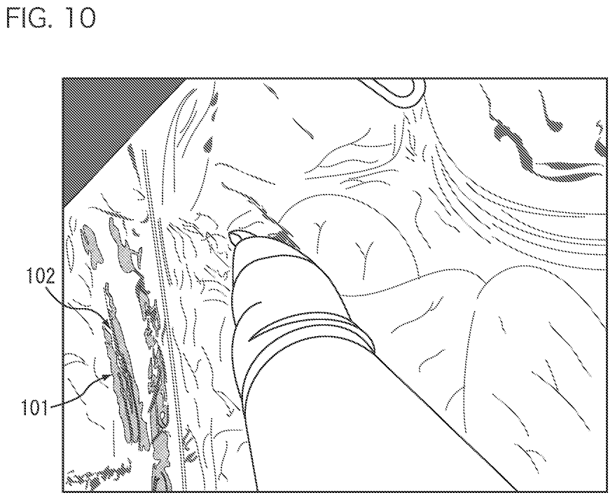

FIG. 10 is a schematic diagram depicting a display example of the display device 130. For the convenience of drawing, in the display example of FIG. 10, a nerve tissue portion 101 recognized by using the learning model 310 is depicted as a hatched region. In practice, the nerve tissue portion 101 is painted with a specific color, such as a white color or a blue color, in units of pixels. Therefore, by viewing the display screen of the display device 130, the operator can clearly recognize the nerve tissue portion 101 so as to be distinguished from a blood vessel tissue portion 102.

In the present embodiment, pixels corresponding to the nerve tissue are displayed so as to be colored with a white or blue color. However, the display color (white or blue color) set in advance and the display color of the background operative field image may be averaged, and the nerve tissue may be displayed so as to be colored with the averaged color. For example, assuming that the display color set for the nerve tissue portion is (R1, G1, B1) and the display color of the nerve tissue portion in the background operative field image is (R2, G2, B2), the control unit 201 may display the recognized nerve tissue portion by coloring the recognized nerve tissue portion with a color ((R1+R2)/2, (G1+G2)/2, (B1+B2)/2). Alternatively, weighting factors W1 and W2 may be introduced, and the recognized blood vessel portion may be displayed so as to be colored with a color (W1×R1+W2×R2, W1×G1+W2×G2, W1×B1+W2×B2).

In addition, the recognized target tissue portion (nerve tissue portion in the present embodiment) may be blinked. That is, the control unit 201 may perform periodic switching between the display and non-display of the target tissue portion by alternately and repeatedly performing processing for displaying the recognized target tissue portion for a first set time (for example, two seconds) and processing for non-displaying the recognized target tissue portion for a second set time (for example, two seconds). The display time and non-display time of the target tissue portion may be set as appropriate. In addition, switching between the display and non-display of the target tissue portion may be performed in synchronization with biological information such as the heartbeat or pulse of the patient. In addition, instead of blinking the target tissue portion, the blood vessel tissue portion may be blinked. By blinking only the target tissue portion excluding the blood vessel tissue portion or by blinking only the blood vessel tissue portion, the target tissue portion can be highlighted so as to be distinguished from the blood vessel tissue portion.

In the flowchart depicted in FIG. 9, when the nerve tissue included in the operative field image is recognized, the recognition image of the nerve tissue portion is displayed so as to be superimposed on the operative field image. However, the superimposed display may be performed only when there is a display instruction. The display instruction may be given through the operation unit 203 of the support apparatus 200, or may be given through the operation portion 11C of the laparoscope 11. Alternatively, the display instruction may be given through a foot switch or the like (not depicted).

In addition, in the present embodiment, the recognition image of the nerve tissue is displayed so as to be superimposed on the operative field image. However, the operator may be notified of the detection of the nerve tissue by sound or voice.

In addition, the control unit 201 of the support apparatus 200 may be configured to generate a control signal for controlling a medical device, such as the energy treatment instrument 12 or a surgical robot (not depicted), based on the nerve tissue recognition result and output the generated control signal to the medical device.

As described above, in the present embodiment, the nerve tissue can be recognized by using the learning model 310, and the recognized nerve tissue can be displayed in a distinguishable manner in units of pixels. Therefore, it is possible to provide visual support in laparoscopic surgery.

When a method of recognizing the nerve tissue and the blood vessel tissue appearing on the surface of the nerve tissue as a single region without distinguishing these from each other is adopted as a nerve recognition method, a region including the nerve in the operative field image is covered with a solid image. Therefore, since the nerve itself becomes difficult to see, there is a possibility that information necessary for the operator who performs the operation will rather be lost.

On the other hand, in the present embodiment, the recognized nerve tissue can be displayed in a distinguishable manner in units of pixels. Therefore, the recognized nerve tissue can be displayed in an easy-to-see manner. In addition, the running directions of nerves are highlighted by displaying the nerve tissue and the blood vessel tissue appearing on the surface of the nerve tissue so as to be distinguished from each other. The operator can predict the presence of invisible nerves by grasping the running directions of the nerves.

In the present embodiment, in order to recognize the nerve tissue so as to be distinguished from the blood vessel tissue appearing on the surface of the nerve tissue, the learning model 310 is generated by performing annotation separately for pixels corresponding to the nerve tissue and pixels corresponding to the blood vessel tissue appearing on the surface of the nerve tissue and performing training by using the training data obtained by the annotation. The surface blood vessel appearing on the surface of the nerve has a pattern unique to the nerve, and is different from the patterns of surface blood vessels appearing on other organs. By using the training data described above during training, not only the position information of the nerve but also the information of the pattern of the surface blood vessel appearing on the surface of the nerve is taken into consideration for training. Therefore, the nerve recognition accuracy is improved.

In addition, the images generated by the support apparatus 200 may be used not only for supporting surgery but also for supporting training of trainees or for evaluating laparoscopic surgery. For example, by determining whether or not a traction operation or a dissection operation in laparoscopic surgery is appropriate by comparing the image generated by the support apparatus 200 with the image recorded in the image recording device 140 during surgery, it is possible to evaluate the laparoscopic surgery.

### Second Embodiment

In a second embodiment, a configuration will be described in which a nerve tissue running in a first direction and a nerve tissue running in a second direction different from the first direction are recognized so as to be distinguished from each other.

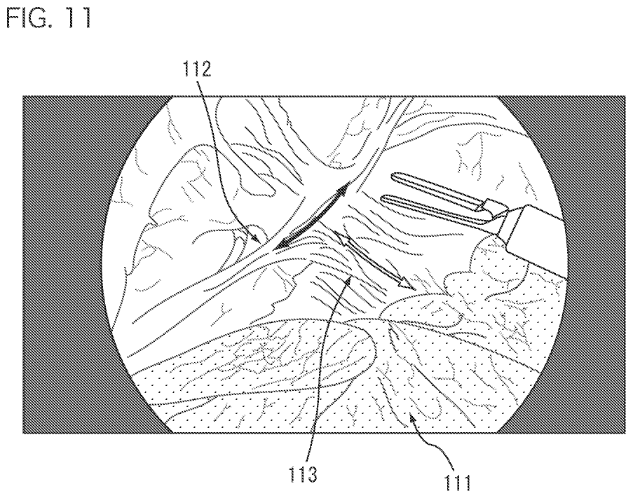

FIG. 11 is a schematic diagram depicting an example of an operative field image in the second embodiment. FIG. 11 depicts an operative field image including an organ 111 appearing in the lower region (dotted region) of the operative field image, a nerve tissue 112 running in a direction along the organ 111 (direction of the black arrow in the diagram), and a nerve tissue 113 that branches from the nerve tissue 112 and runs in a direction toward the organ 111 (direction of the white arrow in the diagram).

Hereinafter, the nerve tissue running along the organ is described as a first nerve tissue, and the nerve tissue running toward the organ is described as a second nerve tissue. In the present embodiment, the first nerve tissue represents a nerve to be preserved in laparoscopic surgery. For example, the vagus nerve, the recurrent laryngeal nerve, or the like corresponds to the first nerve tissue. On the other hand, the second nerve tissue represents a nerve that can be dissected in laparoscopic surgery, and is dissected as necessary when expanding an organ or excising a lesion. The first nerve tissue and the second nerve tissue do not need to be a single nerve tissue, and may be a tissue such as a nerve plexus or a nerve fiber bundle.

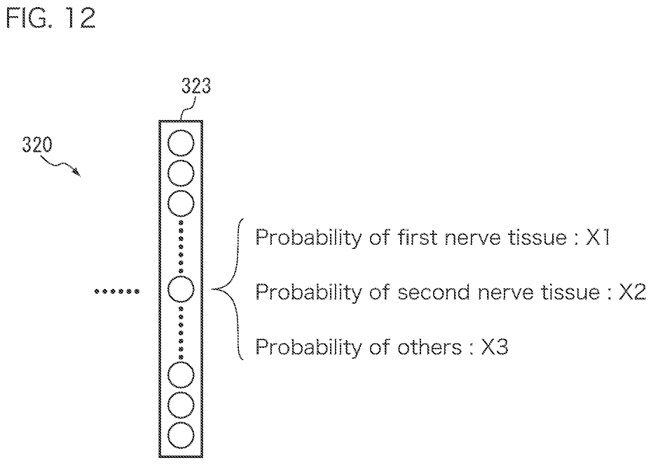

It would be useful for the operator when the first nerve tissue running in one direction (referred to as the first direction) and the second nerve tissue running in the other direction (referred to as the second direction) can be recognized so as to be distinguished from each other and the recognition result can be provided to the operator. Therefore, the support apparatus 200 according to the second embodiment recognizes the first nerve tissue running in the first direction so as to be distinguished from the second nerve tissue running in the second direction by using a learning model 320 (see FIG. 12).

FIG. 12 is an explanatory diagram illustrating the configuration of the learning model 320 according to the second embodiment. FIG. 12 depicts only a softmax layer 323 of the learning model 320 for simplification. Configurations other than the softmax layer 323 are the same as those of the learning model 310 depicted in the first embodiment. The softmax layer 323 included in the learning model 320 according to the second embodiment outputs a probability for a label set corresponding to each pixel. In the second embodiment, a label for identifying the first nerve tissue, a label for identifying the second nerve tissue, and a label indicating something else are set. The control unit 201 of the support apparatus 200 recognizes that the pixel is the first nerve tissue when the probability of the label for identifying the first nerve tissue is equal to or greater than a threshold value, and recognizes that the pixel is the second nerve tissue when the probability of the label for identifying the second nerve tissue is equal to or greater than the threshold value. In addition, the control unit 201 recognizes that the pixel is neither the first nerve tissue nor the second nerve tissue when the probability of the label indicating something else is equal to or greater than the threshold value.

The learning model 320 for obtaining such a recognition result is generated by learning a set including an operative field image and correct data indicating the respective positions (pixels) of the first nerve tissue and the second nerve tissue included in the operative field image using training data. Since the method of generating the learning model 320 is the same as that in the first embodiment, the description thereof will be omitted.

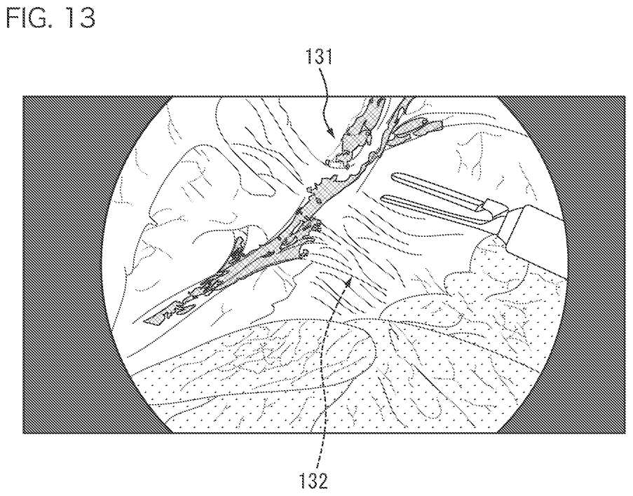

FIG. 13 is a schematic diagram depicting a display example of a recognition result in the second embodiment. The control unit 201 of the support apparatus 200 acquires the recognition result of the learning model 320 by inputting the operative field image to the learning model 320 that has completed training. The control unit 201 generates a recognition image in which the first nerve tissue and the second nerve tissue can be distinguished from each other by referring to the recognition result of the learning model 320. For example, the control unit 201 can generate a recognition image by assigning a specific color, such as a white color or a blue color, to pixels recognized as the first nerve tissue and assigning a different color to pixels recognized as the second nerve tissue.

In the display example of FIG. 13, for convenience of drawing, only a first nerve tissue portion 131 (nerve tissue portion running along the organ) is hatched. In practice, the portion corresponding to the first nerve tissue may be displayed with a specific color, such as a white color or a blue color, in units of pixels.

In addition, although only the first nerve tissue portion 131 is displayed in FIG. 13, only a second nerve tissue portion 132 (nerve tissue portion running in the direction toward the organ) may be displayed, or the first nerve tissue portion 131 and the second nerve tissue portion 132 may be displayed in different display modes.

As described above, in the second embodiment, the nerve tissue running in the first direction and the nerve tissue running in the second direction can be recognized so as to be distinguished from each other. Therefore, for example, the operator can see the presence of the nerve tissue to be preserved and the presence of the nerve tissue that can be dissected.

In addition, although both the nerve tissue running in the first direction and the nerve tissue running in the second direction are recognized in the present embodiment, only the nerve tissue running in the first direction (or the second direction) may be recognized. In this case, the learning model 320 may be generated by using training data including pixels corresponding to the nerve tissue running in the first direction (or the second direction) as correct data and pixels corresponding to the nerve tissue running in the second direction (or the first direction) as incorrect data. By recognizing the nerve tissue using such a learning model 320, it is possible to recognize only the nerve tissue running in the first direction (or the second direction).

### Third Embodiment

In a third embodiment, a configuration will be described in which a nerve tissue and a loose connective tissue are recognized so as to be distinguished from each other.

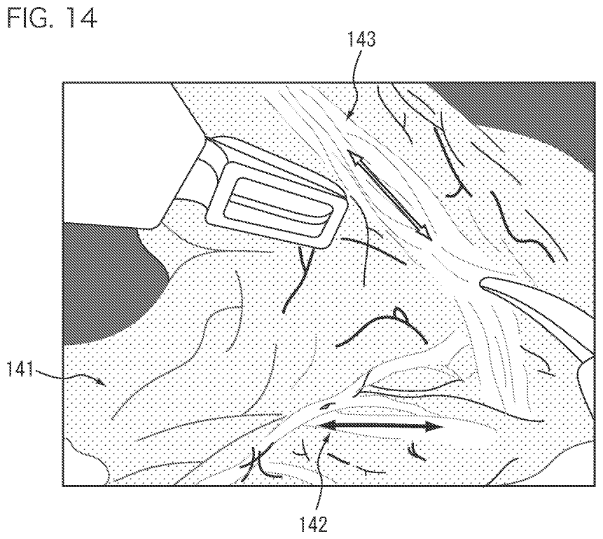

FIG. 14 is a schematic diagram depicting an example of an operative field image in the third embodiment. FIG. 14 depicts an operative field image including an organ 141 appearing in the central region (dotted region) of the operative field image, a nerve tissue 142 running in the horizontal direction (direction of the black arrow in the diagram) on the surface of the organ 141, and a loose connective tissue 143 running in a direction (direction of the white arrow in the diagram) crossing the nerve tissue 142.

The loose connective tissue is a fibrous connective tissue that fills between tissues or organs, and has a relatively small amount of fibers (collagen fibers or elastic fibers) forming the tissue. The loose connective tissue is dissected as necessary when expanding an organ or when excising a lesion.

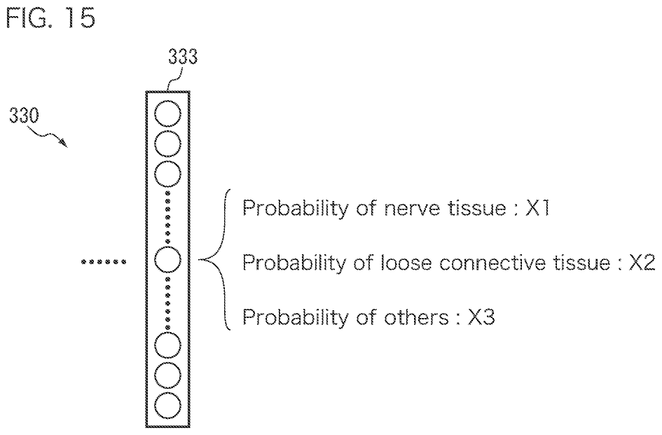

Since both the nerve tissue and the loose connective tissue appearing in the operative field image are white and extend linearly, it is often difficult to visually distinguish the nerve tissue and the loose connective tissue from each other. For this reason, it would be useful for the operator when the nerve tissue and the loose connective tissue can be recognized so as to be distinguished from each other and the recognition result can be provided to the operator. Therefore, the support apparatus 200 according to the third embodiment recognizes the nerve tissue so as to be distinguished from the loose connective tissue by using a learning model 330 (see FIG. 15).

FIG. 15 is an explanatory diagram illustrating the configuration of the learning model 330 according to the third embodiment. FIG. 15 depicts only a softmax layer 333 of the learning model 330 for simplification. Configurations other than the softmax layer 333 are the same as those of the learning model 310 depicted in the first embodiment. The softmax layer 333 included in the learning model 330 according to the third embodiment outputs a probability for a label set corresponding to each pixel. In the third embodiment, a label for identifying the nerve tissue, a label for identifying the loose connective tissue, and a label indicating something else are set. The control unit 201 of the support apparatus 200 recognizes that the pixel is the nerve tissue when the probability of the label for identifying the nerve tissue is equal to or greater than a threshold value, and recognizes that the pixel is the loose connective tissue when the probability of the label for identifying the loose connective tissue is equal to or greater than the threshold value. In addition, the control unit 201 recognizes that the pixel is neither the nerve tissue nor the loose connective tissue when the probability of the label indicating something else is equal to or greater than the threshold value.

The learning model 330 for obtaining such a recognition result is generated by training a set including an operative field image and correct data indicating the respective positions (pixels) of the nerve tissue and the loose connective tissue included in the operative field image using training data. Since the method of generating the learning model 330 is the same as that in the first embodiment, the description thereof will be omitted.

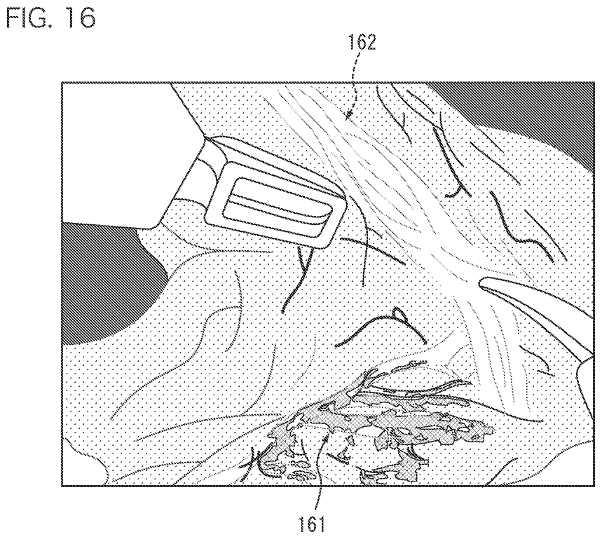

FIG. 16 is a schematic diagram depicting a display example of a recognition result in the third embodiment. The control unit 201 of the support apparatus 200 acquires the recognition result of the learning model 330 by inputting the operative field image to the learning model 330 that has completed training. The control unit 201 generates a recognition image in which the nerve tissue and the loose connective tissue can be distinguished from each other by referring to the recognition result of the learning model 330. For example, the control unit 201 can generate a recognition image by assigning a specific color, such as a white color or a blue color, to pixels recognized as the nerve tissue and assigning a different color to pixels recognized as the loose connective tissue.

In the display example of FIG. 16, for convenience of drawing, only a nerve tissue portion 161 is hatched. In practice, the portion corresponding to the nerve tissue may be displayed with a specific color, such as a white color or a blue color, in units of pixels.

In addition, although only the nerve tissue portion 161 is displayed in FIG. 16, only a loose connective tissue portion 162 may be displayed, or the nerve tissue portion 161 and the loose connective tissue portion 162 may be displayed in different display modes.

As described above, in the third embodiment, the nerve tissue and the loose connective tissue can be recognized so as to be distinguished from each other. Therefore, for example, the operator can see the presence of the nerve tissue to be preserved and the presence of the loose connective tissue that can be dissected.

In addition, although both the nerve tissue and the loose connective tissue are recognized in the present embodiment, only the nerve tissue (or the loose connective tissue) may be recognized. In this case, the learning model 330 may be generated by using training data including pixels corresponding to the nerve tissue (or the loose connective tissue) as correct data and pixels corresponding to the loose connective tissue (or the nerve tissue) as incorrect data. By using such a learning model 330, the control unit 201 can recognize only the nerve tissue (or the loose connective tissue) so as to be distinguished from the loose connective tissue (or the nerve tissue).

### Fourth Embodiment

In a fourth embodiment, a configuration will be described in which the display mode is changed according to the confidence of a nerve tissue recognition result.

As described in the first embodiment, the softmax layer 313 of the learning model 310 outputs a probability for the label set corresponding to each pixel. This probability represents the confidence of the recognition result. The control unit 201 of the support apparatus 200 changes the display mode of the nerve tissue portion according to the confidence of the recognition result.

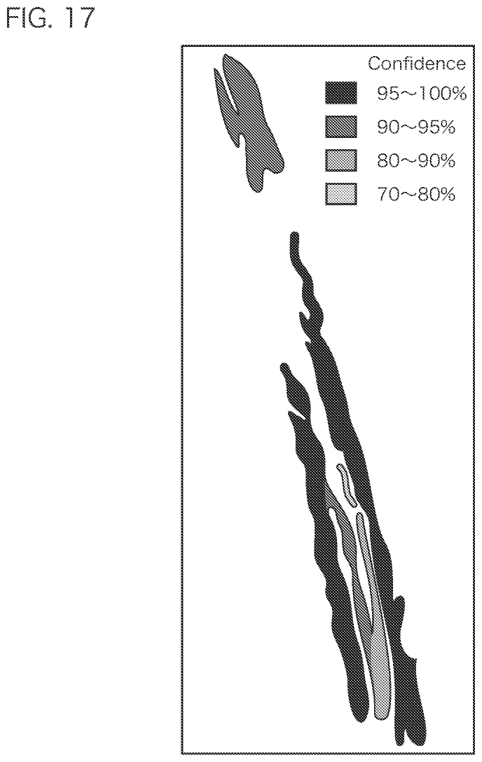

FIG. 17 is a schematic diagram depicting a display example in the fourth embodiment. FIG. 17 depicts a region including a nerve tissue in an enlarged manner. In this example, a nerve tissue portion is displayed with different concentrations for cases where the confidence of the nerve tissue recognition result is 70% to 80%, 80% to 90%, 90% to 95%, and 95% to 100%. In this example, the display mode is changed so that the concentration increases as the confidence increases.

In addition, although the concentration is changed according to the confidence in the example of FIG. 17, the color or the degree of transparency may be changed according to the confidence. When the color is changed, for example, the whiter color may be displayed as the confidence becomes lower, and the bluer color may be displayed as the confidence becomes higher. In addition, when the degree of transparency is changed, the display mode may be changed so that the degree of transparency decreases as the confidence increases.

In addition, although the concentration is changed in four stages according to the confidence in the example of FIG. 17, the concentration may be set more finely and gradation display according to the confidence may be performed.

### Fifth Embodiment

In a fifth embodiment, a configuration will be described in which the estimated position of a nerve tissue portion that is hidden behind an object, such as a surgical instrument, and cannot be visually recognized is displayed.

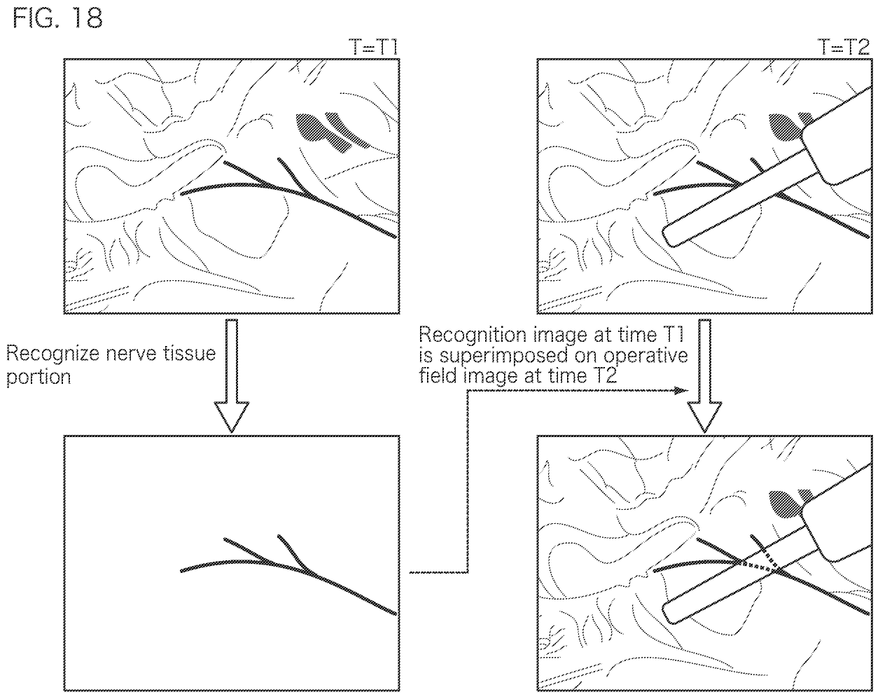

FIG. 18 is an explanatory diagram illustrating a display method according to the fifth embodiment. As described above, the support apparatus 200 recognizes a nerve tissue portion included in the operative field image by using the learning model 310. However, when an object such as a surgical instrument or gauze is present in the operative field to be imaged, the support apparatus 200 cannot recognize the nerve tissue hidden behind the object from the operative field image even when the learning model 310 is used. For this reason, when a recognition image of the nerve tissue is displayed so as to be superimposed on the operative field image, the nerve tissue portion hidden behind the object cannot be displayed in a distinguishable manner.

Therefore, the support apparatus 200 according to the fifth embodiment stores, in the storage unit 202, the recognition image of the nerve tissue recognized in a state in which the nerve tissue is not hidden behind the object, and reads the recognition image stored in the storage unit 202 and displays the read recognition image so as to be superimposed on the operative field image when the nerve tissue portion is hidden behind the object.

In the example of FIG. 18, time T1 depicts an operative field image in a state in which the nerve tissue is not hidden behind the surgical instrument, and time T2 depicts an operative field image in a state in which a part of the nerve tissue is hidden behind the surgical instrument. However, it is assumed that, between time T1 and time T2, the laparoscope 11 is not moved and there is no change in the imaged region.

From the operative field image at time T1, it is possible to recognize the nerve tissue appearing in the operative field. Therefore, a recognition image of the nerve tissue is generated from the recognition result of the learning model 310. The generated recognition image of the nerve tissue is stored in the storage unit 202.

On the other hand, from the operative field image at time T2, the nerve tissue that is not hidden by the surgical instrument, among the nerve tissues appearing in the operative field, can be recognized, but the nerve tissue that is hidden by the surgical instrument cannot be recognized. Therefore, the support apparatus 200 displays the recognition image of the nerve tissue generated from the operative field image at time T1 so as to be superimposed on the operative field image at time T2. In the example of FIG. 18, a portion indicated by the dashed line is a nerve tissue portion that is hidden by the surgical instrument and cannot be visually recognized. However, the support apparatus 200 can display an image including the portion in a distinguishable manner by using the recognition image recognized at time T1.

As described above, in the fifth embodiment, it is possible to notify the operator of the presence of the nerve tissue that is hidden behind the object, such as a surgical instrument or gauze, and cannot be visually recognized. Therefore, it is possible to improve safety during surgery.

In the present embodiment, the nerve tissue portion hidden behind the object is displayed in a distinguishable manner by using the recognition image of the nerve tissue recognized in a state in which the nerve tissue is not hidden behind the object. However, the support apparatus 200 may display the nerve tissue portion in a distinguishable manner by estimating the nerve tissue portion hidden behind the object using a mathematical method, such as interpolation or extrapolation. In addition, the support apparatus 200 may display the nerve tissue portion that is not hidden behind the object and the nerve tissue portion hidden behind the object in different display modes (different colors, concentrations, degrees of transparency, and the like). In addition, the support apparatus 200 may generate a recognition image including both the nerve tissue portion that is not hidden behind the object and the nerve tissue portion hidden behind the object by using a learning model of an image generation system, such as a GAN (Generative Adversarial Network) or a VAE (Variational AutoEncoder), and display the generated recognition image so as to be superimposed on the operative field image.

### Sixth Embodiment

In a sixth embodiment, a configuration will be described in which the running pattern of the nerve tissue is predicted and a nerve portion estimated from the predicted running pattern of the nerve tissue is displayed in a distinguishable manner.

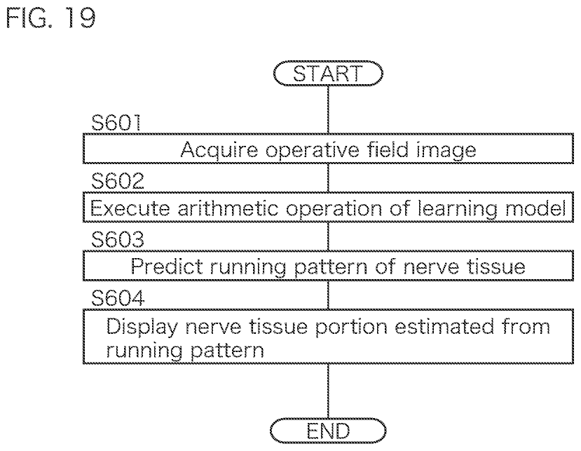

FIG. 19 is a flowchart illustrating the procedure of processing performed by the support apparatus 200 according to the sixth embodiment. As in the first embodiment, the control unit 201 of the support apparatus 200 acquires an operative field image (step S601), and inputs the acquired operative field image to the learning model 310 to execute the arithmetic operation of the learning model 310 (step S602). The control unit 201 predicts the running pattern of the nerve tissue based on the arithmetic result of the learning model 310 (step S603). In the first embodiment, the recognition image of the nerve tissue portion is generated by extracting pixels for which the probability of the label output from the softmax layer 313 of the learning model 310 is equal to or greater than a threshold value (for example, 70% or more). In the sixth embodiment, however, the running pattern of the nerve tissue is predicted by reducing the threshold value. Specifically, the control unit 201 predicts the running pattern of the nerve tissue by extracting pixels for which the probability of the label output from the softmax layer 313 of the learning model 310 is equal to or greater than a first threshold value (for example, 40% or more) and lower than a second threshold value (for example, under 70%).

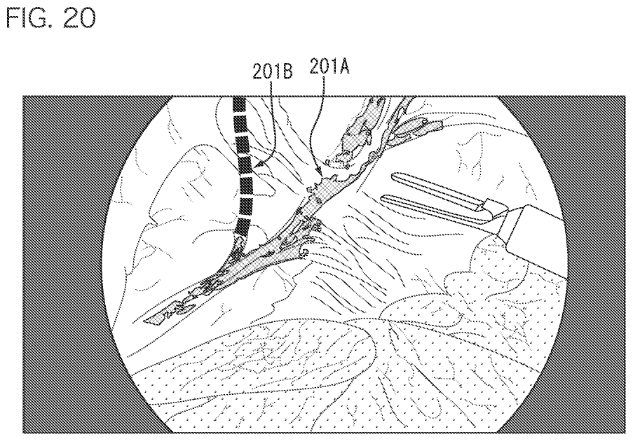

The control unit 201 displays the nerve tissue portion estimated from the predicted running pattern in a distinguishable manner (step S604). FIG. 20 is a schematic diagram depicting a display example according to the sixth embodiment. In FIG. 20, a recognized nerve tissue portion 201A is indicated by hatching, and a nerve tissue portion 201B estimated by the predicted running pattern is indicated by a thick dashed line. For convenience of drawing, in the example of FIG. 20, the recognized nerve tissue portion 201A is indicated by hatching, and the nerve tissue portion 201B estimated from the running pattern is indicated by a thick dashed line. However, the recognized nerve tissue portion 201A and the nerve tissue portion 201B estimated from the running pattern may be displayed in different display modes (different colors, concentrations, degrees of transparency, and the like).

As described above, in the sixth embodiment, since the nerve tissue portion estimated from the running pattern can also be displayed, it is possible to perform visual support in laparoscopic surgery.

In the present embodiment, the running pattern of the nerve tissue is predicted by reducing the threshold value when recognizing the nerve tissue. However, the support apparatus 200 may generate a recognition image including the running pattern of the nerve tissue that cannot be clearly recognized from the operative field image by using a learning model of an image generation system, such as a GAN or a VAE, and display the generated recognition image so as to be superimposed on the operative field image.

### Seventh Embodiment

In the first to sixth embodiments, the configurations in which the nerve tissue is recognized as a target tissue have been described. However, the target tissue is not limited to the nerve tissue, and may be a ureter. In a seventh embodiment, a configuration for recognizing a ureter instead of the nerve tissue will be described.

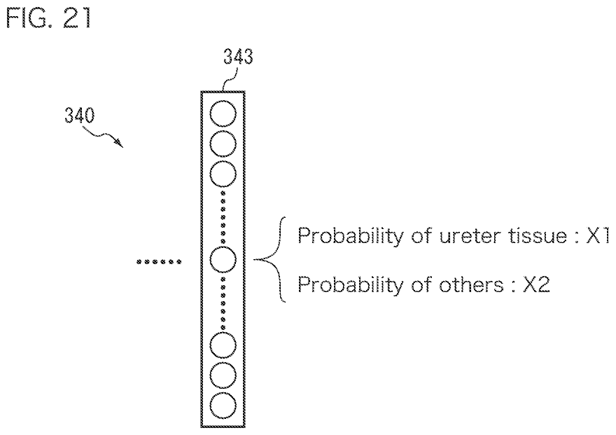

FIG. 21 is an explanatory diagram illustrating the configuration of a learning model 340 according to the seventh embodiment. FIG. 21 depicts only a softmax layer 343 of the learning model 340 for simplification. Configurations other than the softmax layer 343 are the same as those of the learning model 310 depicted in the first embodiment. The softmax layer 343 included in the learning model 340 according to the seventh embodiment outputs a probability for a label set corresponding to each pixel. In the seventh embodiment, a label for identifying the ureter tissue and a label indicating something else are set. The control unit 201 of the support apparatus 200 recognizes that the pixel is the ureter tissue when the probability of the label for identifying the ureter tissue is equal to or greater than a threshold value. In addition, the control unit 201 recognizes that the pixel is not the ureter tissue when the probability of the label indicating something else is equal to or greater than the threshold value.

The learning model 340 for obtaining such a recognition result is generated by training a set including an operative field image and correct data indicating the position (pixel) of the ureter tissue included in the operative field image using training data. That is, the learning model 340 according to the seventh embodiment is trained so as to recognize the ureter tissue and the blood vessel tissue in a distinguishable manner. Since the method of generating the learning model 340 is the same as that in the first embodiment, the description thereof will be omitted.

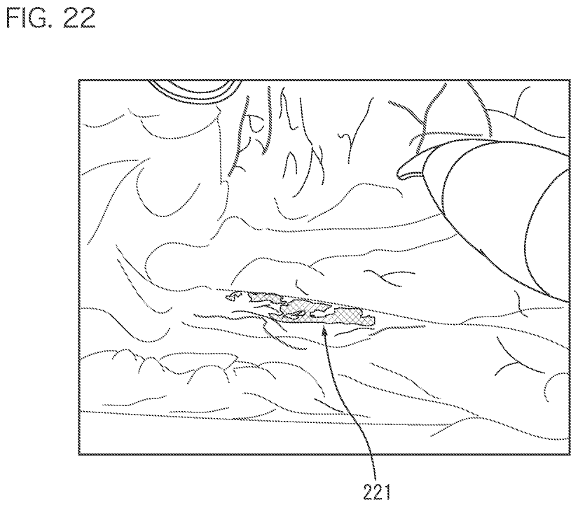

FIG. 22 is a schematic diagram depicting a display example of a recognition result in the seventh embodiment. The control unit 201 of the support apparatus 200 acquires the recognition result of the learning model 340 by inputting the operative field image to the learning model 340 that has completed training. The control unit 201 generates a recognition image in which the ureter tissue and other tissues including the blood vessel tissue can be distinguished from each other by referring to the recognition result of the learning model 340. For example, the control unit 201 can generate a recognition image by assigning a specific color, such as a white color or a blue color, to pixels recognized as the ureter tissue.

In the display example of FIG. 22, for convenience of drawing, only a ureter tissue portion 221 is hatched. In practice, the portion corresponding to the ureter tissue may be displayed with a specific color, such as a white color or a blue color, in units of pixels.

As described above, in the seventh embodiment, the ureter tissue can be recognized by using the learning model 340, and the recognized ureter tissue can be displayed in a distinguishable manner in units of pixels. Therefore, it is possible to provide visual support in laparoscopic surgery.

When a method of recognizing the ureter tissue and the blood vessel tissue appearing on the surface of the ureter tissue as a single region without distinguishing these from each other is adopted as a ureter recognition method, a region including the ureter in the operative field image is covered with a solid image. Therefore, since the ureter itself becomes difficult to see, there is a possibility that information necessary for the operator who performs the operation will rather be lost. For example, the ureter performs peristalsis to carry urine from the renal pelvis to the bladder, but it may be difficult to recognize the peristalsis when the region including the ureter is covered with a solid image.

On the other hand, in the present embodiment, the recognized ureter tissue can be displayed in a distinguishable manner in units of pixels. Therefore, the recognized ureter tissue can be displayed in an easy-to-see manner. In particular, in the present embodiment, since the ureter tissue and the blood vessel tissue (surface blood vessel) appearing on the surface of the ureter tissue are displayed so as to be distinguished from each other, the presence of the surface blood vessel that moves with the peristalsis of the ureter is highlighted. As a result, the operator can easily recognize the peristalsis of the ureter. In addition, by performing display so that the blood vessel tissue appearing on the surface of the ureter tissue is excluded, the running direction of the ureter is highlighted. The operator can predict the presence of the invisible ureter by grasping the running direction of the ureter.

In the present embodiment, in order to recognize the ureter tissue so as to be distinguished from the blood vessel tissue appearing on the surface of the ureter tissue, the learning model 340 is generated by performing annotation separately for pixels corresponding to the ureter tissue and pixels corresponding to the blood vessel tissue appearing on the surface of the ureter tissue and performing training by using the training data obtained by the annotation. The surface blood vessel appearing on the surface of the ureter has a pattern unique to the ureter, and is different from the patterns of surface blood vessels appearing on other organs. By using the training data described above during training, not only the position information of the ureter but also the information of the pattern of the surface blood vessel appearing on the surface of the ureter is taken into consideration for learning. Therefore, the ureter recognition accuracy is training.

### Eighth Embodiment

In an eighth embodiment, a configuration will be described in which surface blood vessels appearing on the surface of an organ are recognized and the end position of the recognized surface blood vessel portion is specified to specify the boundary of the organ.

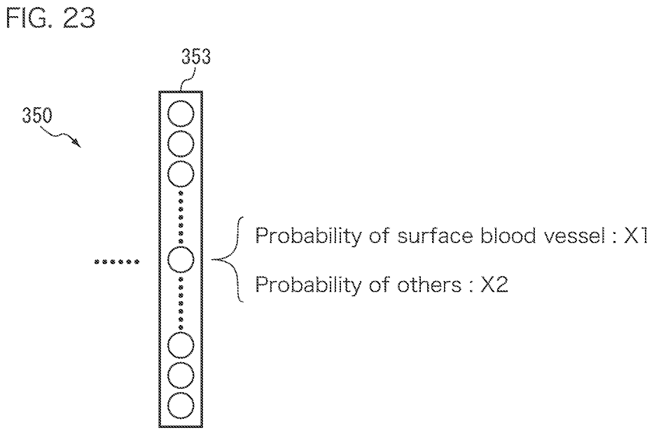

FIG. 23 is an explanatory diagram illustrating the configuration of a learning model 350 according to the eighth embodiment. FIG. 23 depicts only a softmax layer 353 of the learning model 350 for simplification. Configurations other than the softmax layer 353 are the same as those of the learning model 310 depicted in the first embodiment. The softmax layer 353 included in the learning model 350 according to the eighth embodiment outputs a probability for a label set corresponding to each pixel. In the eighth embodiment, a label for identifying a surface blood vessel and a label indicating something else are set. The control unit 201 of the support apparatus 200 recognizes that the pixel is a pixel corresponding to the surface blood vessel when the probability of the label for identifying the surface blood vessel is equal to or greater than a threshold value. In addition, the control unit 201 recognizes that the pixel is not the surface blood vessel when the probability of the label indicating something else is equal to or greater than the threshold value.

The learning model 350 for obtaining such a recognition result is generated by training a set including an operative field image and correct data indicating the position (pixel) of the surface blood vessel included in the operative field image using training data. That is, the learning model 350 according to the eighth embodiment is trained so as to recognize the surface blood vessel and other tissues in a distinguishable manner. Since the method of generating the learning model 350 is the same as that in the first embodiment, the description thereof will be omitted.

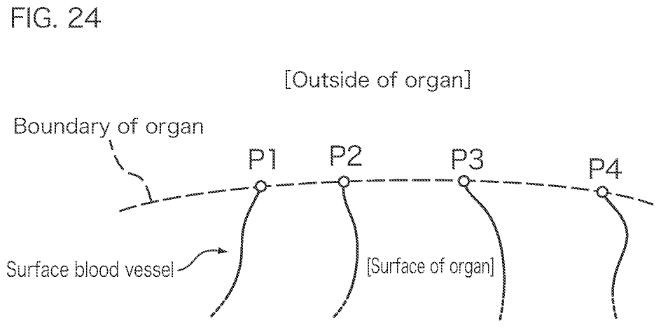

FIG. 24 is an explanatory diagram illustrating a method of specifying the organ boundary. The control unit 201 of the support apparatus 200 acquires the recognition result of the learning model 350 by inputting the operative field image to the learning model 350 that has completed training. The control unit 201 generates a recognition image of the surface blood vessel appearing on the surface of the organ by referring to the recognition result of the learning model 350. The solid line in FIG. 24 indicates the surface blood vessel recognized by the learning model 350.

The control unit 201 specifies the position coordinates of the end of the surface blood vessel in the generated recognition image. For example, the control unit 201 can specify the position coordinates of the end by calculating, for each of pixels forming the segment of the surface blood vessel, the number of adjacent pixels belonging to the same segment and specifying pixels for which the number of adjacent pixels is 1. FIG. 24 depicts an example in which the coordinates of four points P1 to P4 are specified as the position coordinates of the end of the surface blood vessel. The control unit 201 specifies the boundary of the organ where the surface blood vessel appears by deriving an approximate curve passing through the specified points P1 to P4 (or the vicinity of the points P1 to P4). A known method, such as a least square method, can be used to derive the approximate curve. In addition, the control unit 201 may specify the boundary of the organ where the surface blood vessel appears by deriving a closed curve including all the specified end points.

In addition, the control unit 201 does not need to specify the entire organ boundary, and may be configured to specify a part of the organ boundary.

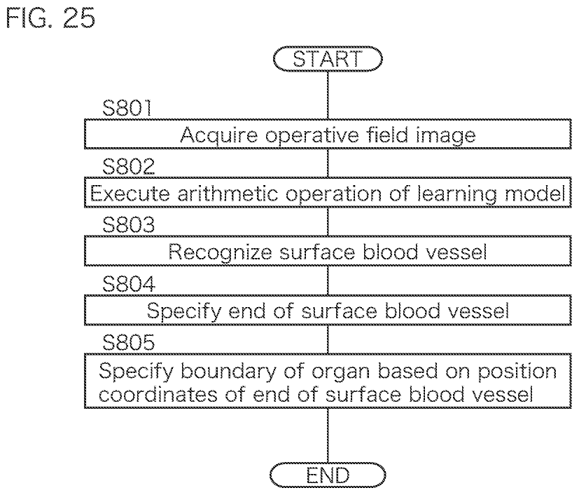

FIG. 25 is a flowchart illustrating the procedure of processing performed by the support apparatus 200 according to the eighth embodiment. As in the first embodiment, the control unit 201 of the support apparatus 200 acquires an operative field image (step S801), and inputs the acquired operative field image to the learning model 350 to execute the arithmetic operation of the learning model 350 (step S802). The control unit 201 recognizes the surface blood vessel appearing on the surface of the organ based on the arithmetic result of the learning model 350 (step S803).

Then, the control unit 201 specifies the position coordinates of the end of the surface blood vessel (step S804). At this time, the control unit 201 may specify the position coordinates of the ends of all surface blood vessels, or may extract only the surface blood vessel whose length is equal to or greater than a threshold value and specify the position coordinates of the end thereof.

Then, the control unit 201 specifies the boundary of the organ based on the specified position coordinates of the end of the surface blood vessel (step S805). As described above, the control unit 201 can specify the boundary of the organ where the surface blood vessel appears by deriving an approximate curve passing through the specified position coordinates (or the vicinity of the position coordinates) of the end of the surface blood vessel.

As described above, in the eighth embodiment, the boundary of an organ can be specified by using surface blood vessels appearing on the surface of the organ as clues. The support apparatus 200 can support surgery by presenting the information of the specified boundary to the operator.

In the eighth embodiment, the boundary of the organ is specified. However, the target tissue whose boundary is to be specified is not limited to the organ, and may be a membrane, layer, or the like that covers the organ.

It should be considered that the embodiments disclosed are examples in all points and not restrictive. The scope of the present invention is defined by the claims rather than the meanings set forth above, and is intended to include all modifications within the scope and meaning equivalent to the claims.

It is noted that, as used herein and in the appended claims, the singular forms “a”, “an”, and “the” include plural referents unless the context clearly dictates otherwise.

1-16. (canceled)

17. A non-transitory computer readable recording medium storing a computer program that causes a computer to execute processing comprising:
acquiring an operative field image obtained by imaging an operative field of scopic surgery; and
recognizing a target tissue portion included in the acquired operative field image so as to be distinguished from a blood vessel tissue portion appearing on a surface of the target tissue portion by using a learning model trained to output information regarding a target tissue when the operative field image is input.

18. The non-transitory computer readable recording medium according to claim 17, storing the computer program that causes the computer to execute processing comprising:
displaying the target tissue portion and the blood vessel tissue portion so as to be distinguishable from each other on the operative field image.

19. The non-transitory computer readable recording medium according to claim 17, storing the computer program that causes the computer to execute processing comprising:
periodically switching display and non-display of the target tissue portion.

20. The non-transitory computer readable recording medium according to claim 17, storing the computer program that causes the computer to execute processing comprising:
periodically switching display and non-display of the blood vessel tissue portion.

21. The non-transitory computer readable recording medium according to claim 17, wherein
the target tissue is a nerve tissue, and
the computer is caused to execute processing of recognizing the nerve tissue so as to be distinguished from a blood vessel tissue accompanying the nerve tissue by using the learning model.

22. The non-transitory computer readable recording medium according to claim 17, wherein
the target tissue is a nerve tissue running in a first direction, and
the computer is caused to execute processing of recognizing the nerve tissue running in the first direction so as to be distinguished from a nerve tissue running in a second direction different from the first direction by using the learning model.

23. The non-transitory computer readable recording medium according to claim 17, wherein
the target tissue is a nerve tissue, and
the computer is caused to execute processing of recognizing the nerve tissue so as to be distinguished from a loose connective tissue running in a direction crossing the nerve tissue by using the learning model.

24. The non-transitory computer readable recording medium according to claim 17, wherein
the target tissue is a ureter tissue, and
the computer is caused to execute processing of recognizing the ureter tissue so as to be distinguished from a blood vessel tissue accompanying the ureter tissue by using the learning model.

25. The non-transitory computer readable recording medium according to claim 17, storing the computer program that causes the computer to execute processing comprising:
recognizing a target tissue in a tense state included in the operative field image by using the learning model.

26. The non-transitory computer readable recording medium according to claim 17, storing the computer program that causes the computer to execute processing comprising:
calculating a confidence of a recognition result of the learning model; and
displaying the target tissue portion in a display mode according to the calculated confidence.

27. The non-transitory computer readable recording medium according to claim 17, storing the computer program that causes the computer to execute processing comprising:
displaying an estimated position of a target tissue portion hidden behind another object by referring to a recognition result of the learning model.

28. The non-transitory computer readable recording medium according to claim 17, storing the computer program that causes the computer to execute processing comprising:
estimating a running pattern of a target tissue by using the learning model; and
displaying an estimated position of a target tissue portion that does not appear in the operative field image based on the estimated running pattern of the target tissue.

29. A non-transitory computer readable recording medium storing a computer program that causes a computer to execute processing comprising:
acquiring an operative field image obtained by imaging an operative field of scopic surgery;
recognizing a surface blood vessel portion of a target tissue included in the acquired operative field image so as to be distinguished from other tissue portions by using a learning model trained to output information regarding a surface blood vessel of the target tissue when the operative field image is input; and
specifying a boundary of the target tissue by specifying a position of an end of the recognized surface blood vessel portion.

30. A learning model generation method that is executed by a computer, the method comprising:
causing a computer to acquire training data including an operative field image obtained by imaging an operative field of scopic surgery and correct data in which a target tissue portion included in the operative field image is labeled so as to be distinguished from a blood vessel tissue portion appearing on a surface of the target tissue portion; and
causing the computer to generate a learning model that outputs information regarding a target tissue based on the acquired set of training data when the operative field image is input.

31. A support apparatus, comprising:
a processor; and
a storage storing instructions causing the processor to execute processing comprising:
acquiring an operative field image obtained by imaging an operative field of scopic surgery;
recognizing a target tissue portion included in the acquired operative field image so as to be distinguished from a blood vessel tissue portion appearing on a surface of the target tissue portion by using a learning model trained to output information regarding a target tissue when the operative field image is input; and
outputting support information regarding the scopic surgery based on a recognition result.

32. The support apparatus according to claim 31, wherein
the processor displays a recognition image indicating the recognized target tissue portion so as to be superimposed on the operative field image.
